(19) United States
(12) Patent Application Publication
(10) Pub. No.: US 2025/0356490 A1
(43) Pub. Date: Nov. 20, 2025

(54) ASSISTANCE DEVICE, OPERATION METHOD OF ASSISTANCE DEVICE, COMPUTER-READABLE RECORDING MEDIUM, MEDICAL SYSTEM, AND LEARNING DEVICE

(71) Applicant: OLYMPUS MEDICAL SYSTEMS CORP., Tokyo (JP)

(72) Inventors: Yasuo TANIGAMI, Tokyo (JP); Yusuke OTSUKA, Kanagawa (JP); Noriko KURODA, Tokyo (JP); Takaaki IGARASHI, Tokyo (JP)

(73) Assignee: OLYMPUS MEDICAL SYSTEMS CORP., Tokyo (JP)

(21) Appl. No.: 19/287,946

(22) Filed: Aug. 1, 2025

Related U.S. Application Data
(63) Continuation of application No. PCT/JP2023/004456, filed on Feb. 9, 2023.

(51) Int. Cl. G06T 7/00; G06T 7/11

(52) U.S. Cl. CPC G06T 7/0012; G06T 7/11; G06T 2207/10024; G06T 2207/10064; G06T 2207/20012; G06T 2207/20041; G06T 2207/30101

(57) ABSTRACT

An assistance device includes: a processor including hardware, the processor being configured to: acquire a fluorescence image obtained by capturing fluorescence produced by irradiating a biological tissue with excitation light, and a narrowband light observation image captured by irradiating the biological tissue with first narrowband light having a wavelength determined according to an absorption rate of hemoglobin; extract a thermally-denatured region from the fluorescence image; extract a blood vessel region from the narrowband light observation image; perform alignment between the fluorescence image and the narrowband light observation image; and output information corresponding to a distance between the thermally-denatured region and the blood vessel region.

### CROSS REFERENCES TO RELATED APPLICATIONS

This application is a continuation of International Application No. PCT/JP2023/004456, filed on Feb. 9, 2023, the entire contents of which are incorporated herein by reference.

### BACKGROUND

### 1. Technical Field

The present disclosure relates to an assistance device, an operation method of the assistance device, a computer-readable recording medium, a medical system, and a learning device.

### 2. Related Art

In the medical field, minimally invasive treatment using an endoscope, a laparoscope, or the like has been widely performed. For example, endoscopic submucosal dissection (ESD) is widely performed as minimally invasive treatment using an endoscope, a laparoscope, or the like.

In the ESD, an energy device such as a high-frequency knife is used to perform thermal treatment such as resection and coagulation of a diseased tissue.

In addition, a technique for estimating a position of a blood vessel in surgery using an endoscope is known (see, for example, JP 2012-130506 A).

### SUMMARY

In some embodiments, an assistance device includes: a processor including hardware, the processor being configured to: acquire a fluorescence image obtained by capturing fluorescence produced by irradiating a biological tissue with excitation light, and a narrowband light observation image captured by irradiating the biological tissue with first narrowband light having a wavelength determined according to an absorption rate of hemoglobin; extract a thermally-denatured region from the fluorescence image; extract a blood vessel region from the narrowband light observation image; perform alignment between the fluorescence image and the narrowband light observation image; and output information corresponding to a distance between the thermally-denatured region and the blood vessel region.

In some embodiments, a method of operating an assistance device includes: extracting a thermally-denatured region from a fluorescence image obtained by capturing fluorescence produced by irradiating a biological tissue with excitation light; extracting a blood vessel region from a narrowband light observation image captured by irradiating the biological tissue with narrowband light having a wavelength determined according to an absorption rate of hemoglobin; performing alignment between the fluorescence image and the narrowband light observation image; and outputting information corresponding to a distance between the thermally-denatured region and the blood vessel region.

In some embodiments, provided is a non-transitory computer-readable recording medium on which an executable program is recorded. The program causes an assistance device to execute: extracting a thermally-denatured region from a fluorescence image obtained by capturing fluorescence produced by irradiating a biological tissue with excitation light; extracting a blood vessel region from a narrowband light observation image captured by irradiating the biological tissue with narrowband light having a wavelength determined according to an absorption rate of hemoglobin; performing alignment between the fluorescence image and the narrowband light observation image; and outputting information corresponding to a distance between the thermally-denatured region and the blood vessel region.

In some embodiments, a medical system includes: a light source configured to irradiate a biological tissue with excitation light and irradiate the biological tissue with narrowband light having a wavelength determined according to an absorption rate of hemoglobin; an endoscope configured to generate a first imaging signal obtained by capturing fluorescence produced by irradiating the biological tissue with the excitation light and a second imaging signal captured by irradiating the biological tissue with the narrowband light; and an image processing device including a processor including hardware, the processor being configured to: generate a fluorescence image from the first imaging signal; generate a narrowband light observation image from the second imaging signal; extract a thermally-denatured region from the fluorescence image; extract a blood vessel region from the narrowband light observation image; perform alignment between the fluorescence image and the narrowband light observation image; and output information corresponding to a distance between the thermally-denatured region and the blood vessel region.

In some embodiments, a learning device includes: a processor comprising hardware, the processor being configured to generate a learned model by performing machine learning using teacher data in which a fluorescence image obtained by capturing fluorescence produced by irradiating a biological tissue with excitation light and a narrowband light observation image captured by irradiating the biological tissue with narrowband light having a wavelength determined according to an absorption rate of hemoglobin are used as input data, alignment between a thermally-denatured region extracted from the fluorescence image and a blood vessel region extracted from the narrowband light observation image is performed, and information according to a distance between the thermally-denatured region and the blood vessel region after performing the alignment is output as output data.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an overall configuration of an endoscope system according to an embodiment;

FIG. 2 is a block diagram illustrating a functional configuration of a main part of the endoscope system according to an embodiment;

FIG. 3 is a flowchart illustrating an outline of processing executed by a control device;

FIG. 4 is a view illustrating an example of a biological tissue of a subject;

FIG. 5 is a diagram illustrating an example of a narrowband light observation image;

FIG. 6 is a view illustrating an example of a fluorescence image;

FIG. 7 is a view illustrating an image created by superimposing a narrowband light observation image and a fluorescence image; and

FIG. 8 is a view illustrating an example of a biological tissue of a subject.

### DETAILED DESCRIPTION

Hereinafter, as a mode for carrying out the present disclosure (hereinafter, referred to as “embodiment”), an endoscope system including an insertion portion with a flexible endoscope will be described, but the present disclosure is not limited thereto, and for example, a rigid endoscope, a surgical robot, and the like can also be applied. In addition, the present disclosure is not limited by this embodiment. Furthermore, in the description of the drawings, the same portions are denoted by the same reference numerals. Furthermore, it should be noted that the drawings are schematic, and the relationship between the thickness and the width of each member, the ratio of each member, and the like are different from reality. In addition, the drawings include portions having different dimensions and ratios from each other.

### Configuration of Endoscope System

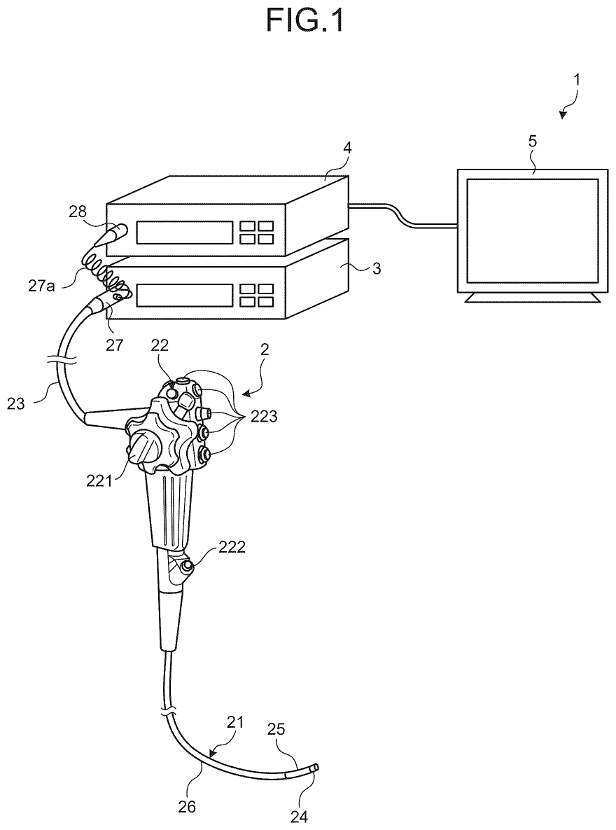

FIG. 1 is a diagram schematically illustrating an overall configuration of an endoscope system according to an embodiment. An endoscope system 1 illustrated in FIG. 1 captures an image of the inside of a body of a subject such as a patient by inserting an insertion portion of an endoscope into a body cavity or a lumen of the subject, and displays a display image based on the captured imaging signal on a display device. The endoscope system 1 includes an endoscope 2, a light source device 3, a control device 4, and a display device 5.

### Configuration of Endoscope

First, a configuration of the endoscope 2 will be described.

The endoscope 2 generates an imaging signal (RAW data) obtained by imaging the inside of the body of the subject, and outputs the generated imaging signal to the control device 4. Specifically, the endoscope 2 generates a first imaging signal obtained by capturing fluorescence produced by irradiating excitation light and a second imaging signal captured by irradiating narrowband light. The endoscope 2 includes an insertion portion 21, an operating unit 22, and a universal cord 23.

The insertion portion 21 is inserted into the subject. The insertion portion 21 has an elongated shape having flexibility. The insertion portion 21 includes a distal end portion 24 incorporating an imaging element described later, a bendable bending portion 25 including a plurality of bending pieces, and an elongated flexible tube portion 26 connected to a proximal end side of the bending portion 25 and having flexibility.

The distal end portion 24 is configured using glass fiber or the like. The distal end portion 24 forms a light guide path of the illumination light supplied from the control device 4 via the universal cord 23 and the operating unit 22, generates an imaging signal obtained by imaging return light of the illumination light, and outputs the imaging signal to the control device 4.

The operating unit 22 includes, in addition to a bending knob 221 that bends the bending portion 25 in the vertical direction and the horizontal direction, a treatment tool insertion portion 222 into which a body treatment tool is inserted, and the control device 4, a plurality of switches 223 that is an operation input unit that inputs an operation instruction signal of a peripheral device such as an air supply unit, a water supply unit, or a gas supply unit, a pre-freeze signal that instructs the endoscope system 1 to capture a still image, or a switching signal that switches an observation mode of the endoscope system 1. The treatment tool inserted from the treatment tool insertion portion 222 comes out from an aperture (not illustrated) via a treatment tool channel (not illustrated) of the distal end portion 24.

The universal cord 23 incorporates at least a light guide and an assembly cable including one or a plurality of cables. The assembly cable is a signal line for transmitting and receiving a signal between the endoscope 2 and the control device 4, and includes a signal line for transmitting and receiving an imaging signal (RAW data) and a signal line for transmitting and receiving a driving timing signal (a synchronization signal and a clock signal) for driving an imaging element to be described later. The universal cord 23 includes a connector portion 27 detachable from the control device 4, and a connector portion 28 in which a coil-shaped coil cable 27a extends and which is detachable from the control device 4 at an extending end of the coil cable 27a.

### Configuration of Light Source Device

Next, a configuration of the light source device will be described.

The light source device 3 irradiates a biological tissue with the excitation light and irradiates the biological tissue with narrowband light having a wavelength determined according to the absorption rate of hemoglobin. The light source device 3 is connected to one end of a light guide of the endoscope 2, and supplies illumination light irradiating the inside of the subject to one end of the light guide under the control of the control device 4. The light source device 3 is realized by using one or more light sources of semiconductor laser elements such as a light emitting diode (LED) light source, a xenon lamp, and a laser diode (LD), a processor that is a processing device having hardware such as a field programmable gate array (FPGA) and a central processing unit (CPU), and a memory that is a temporary storage area used by the processor. Note that the light source device 3 and the control device 4 may be configured to communicate individually as illustrated in FIG. 1, or may be integrated.

Configuration of Control Device Next, a configuration of the control device 4 will be described.

The control device 4 controls each unit of the endoscope system 1. The control device 4 supplies illumination light for the endoscope 2 to irradiate the subject. In addition, the control device 4 performs various types of image processing on the imaging signal input from the endoscope 2 and outputs the imaging signal to the display device 5.

### Configuration of Display Device

Next, a configuration of the display device 5 will be described.

The display device 5 displays a display image based on the video signal input from the control device 4 under the control of the control device 4. The display device 5 is realized by using a display panel such as organic electro luminescence (EL) or liquid crystal.

Functional Configuration of Main Part of Endoscope System

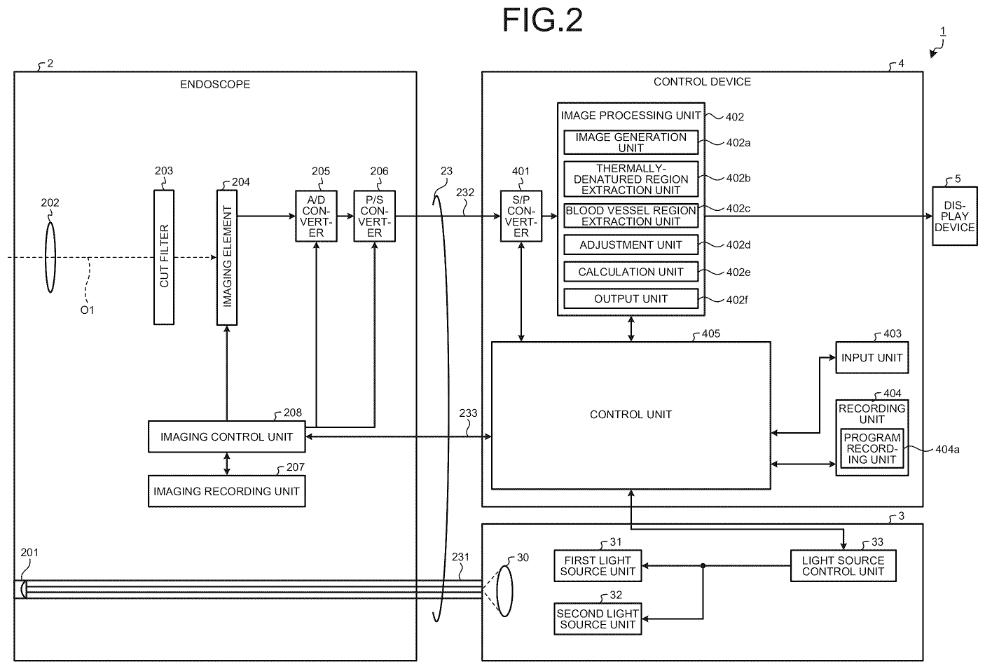

Next, a functional configuration of a main part of the above-described endoscope system 1 will be described. FIG. 2 is a block diagram illustrating a functional configuration of the main part of the endoscope system 1.

### Configuration of Endoscope

First, a configuration of the endoscope 2 will be described.

The endoscope 2 includes an illumination optical system 201, an imaging optical system 202, a cut filter 203, an imaging element 204, an A/D converter 205, a P/S converter 206, an imaging recording unit 207, and an imaging control unit 208. Note that each of the illumination optical system 201, the imaging optical system 202, the cut filter 203, the imaging element 204, the A/D converter 205, the P/S converter 206, the imaging recording unit 207, and the imaging control unit 208 is disposed in the distal end portion 24.

The illumination optical system 201 irradiates a subject (biological tissue) with illumination light supplied from a light guide 231 formed of an optical fiber or the like. The illumination optical system 201 is realized using one or a plurality of lenses.

The imaging optical system 202 condenses light such as reflected light reflected from the subject, return light from the subject, and fluorescence emitted by the subject to form a subject image (light beam) on a light receiving surface of the imaging element 204. The imaging optical system 202 is realized by using one or a plurality of lenses or the like.

The cut filter 203 is disposed on an optical axis O1 between the imaging optical system 202 and the imaging element 204. The cut filter 203 shields light in a wavelength band of reflected light or return light of the excitation light from the subject, which is the excitation light supplied from the control device 4 to be described later, and transmits light in a wavelength band longer than the wavelength band of the excitation light. In addition, the cut filter 203 transmits light in a wavelength band of reflected light or return light of the narrowband light from the subject, which is the narrowband light supplied from the control device 4 described later.

Under the control of the imaging control unit 208, the imaging element 204 receives a subject image (light beam) formed by the imaging optical system 202 and transmitted through the cut filter 203, performs photoelectric conversion, generates an imaging signal (RAW data), and outputs the imaging signal to the A/D converter 205. The imaging element 204 is realized by using a charge coupled device (CCD) or complementary metal oxide semiconductor (CMOS) image sensor in which any one of color filters constituting a Bayer array (RGGB) is arranged in each of a plurality of pixels arranged in a two-dimensional matrix.

Under the control of the imaging control unit 208, the A/D converter 205 performs A/D conversion processing on the analog imaging signal input from the imaging element 204, and outputs the analog imaging signal to the P/S converter 206. The A/D converter 205 is realized by using an A/D conversion circuit or the like.

Under the control of the imaging control unit 208, the P/S converter 206 performs parallel/serial conversion on the digital imaging signal input from the A/D converter 205, and outputs the imaging signal subjected to the parallel/serial conversion to the control device 4 via a first transmission cable 232. The P/S converter 206 is realized by using a P/S conversion circuit or the like. Note that, in the first embodiment, an E/O converter that converts an imaging signal into an optical signal may be provided instead of the P/S converter 206, and the imaging signal may be output to the control device 4 by the optical signal, or the imaging signal may be transmitted to the control device 4 by wireless communication such as Wireless Fidelity (Wi-Fi) (registered trademark), for example.

The imaging recording unit 207 records various types of information regarding the endoscope 2 (for example, pixel information of the imaging element 204 and characteristics of the cut filter 203). Furthermore, the imaging recording unit 207 records various setting data and control parameters transmitted from the control device 4 via a second transmission cable 233. The imaging recording unit 207 is configured using a nonvolatile memory or a volatile memory.

The imaging control unit 208 controls the operation of each of the imaging element 204, the A/D converter 205, and the P/S converter 206 on the basis of the setting data received from the control device 4 via the second transmission cable 233. The imaging control unit 208 is realized by using a timing generator (TG), a processor which is a processing device having hardware such as a CPU, and a memory that is a temporary storage area used by the processor.

### Configuration of Light Source Device

Next, a configuration of the light source device 3 will be described.

The light source device 3 includes a condenser lens 30, a first light source unit 31, a second light source unit 32, and a light source control unit 33.

The condenser lens 30 condenses the light emitted by each of the first light source unit 31 and the second light source unit 32 and emits the light to the light guide 231. The condenser lens 30 includes one or a plurality of lenses.

The first light source unit 31 supplies the narrowband light to the light guide 231 by emitting the narrowband light under the control of the light source control unit 33. The narrowband light is, for example, amber light having a peak wavelength in a wavelength band of 580 nm or more and 620 nm or less, but may be blue-violet light having a peak wavelength in a wavelength band of 390 nm or more and 430 nm or less, or green light having a peak wavelength in a wavelength band of 500 nm or more and 550 nm or less, or may include light in two or more wavelength bands. The first light source unit 31 includes a collimator lens, a light emitting diode (LED) or a laser diode (LD), a drive driver, and the like.

The second light source unit 32 supplies the narrowband light to the light guide 231 as illumination light by emitting excitation light having a predetermined wavelength band under the control of the light source control unit 33. Here, the excitation light has a wavelength at which a substance such as advanced glycation end products (AGEs) contained in the thermally-denatured region is excited, and has, for example, a wavelength band of 400 nm or more and 430 nm or less (central wavelength: 415 nm). The thermally-denatured region is a region where a biological tissue is denatured by heat by thermal treatment performed by an energy device such as a high-frequency knife. The excitation light emitted from the second light source unit 32 is blocked by the cut filter 203, and the fluorescence (wavelength: 540 nm) generated from the AGEs passes through the cut filter 203, so that a fluorescence image can be captured. The second light source unit 32 is realized using a semiconductor laser such as a collimating lens or a violet laser diode (LD), a drive driver, and the like.

The light source control unit 33 includes a processor that is a processing device having hardware such as a field-programmable gate array (FPGA) or a central processing unit (CPU), and a memory that is a temporary storage area used by the processor. The light source control unit 33 controls light emission timing, light emission intensity, light emission time, and the like of each of the first light source unit 31 and the second light source unit 32 on the basis of control data input from a control unit 405.

### Configuration of Control Device

Next, a configuration of the control device 4 will be described.

The control device 4 includes an S/P converter 401, an image processing unit 402, an input unit 403, a recording unit 404, and a control unit 405.

Under the control of the control unit 405, the S/P converter 401 performs serial/parallel conversion on the imaging signal received from the endoscope 2 via the first transmission cable 232 and outputs the imaging signal to the image processing unit 402. Note that, in a case where the endoscope 2 outputs an imaging signal as an optical signal, an O/E converter that converts the optical signal into an electric signal may be provided instead of the S/P converter 401. Furthermore, in a case where the endoscope 2 transmits an imaging signal by wireless communication, a communication module capable of receiving a wireless signal may be provided instead of the S/P converter 401.

The image processing unit 402 is realized by using a processor having hardware such as a CPU, a graphics processing unit (GPU), or an FPGA, and a memory that is a temporary storage area used by the processor. Under the control of the control unit 405, the image processing unit 402 performs predetermined image processing on the imaging signal input from the S/P converter 401 and outputs the imaging signal to the display device 5. Note that, in an embodiment, the image processing unit 402 functions as an assistance device and an image processing device. The image processing unit 402 generates a fluorescence image from the first imaging signal and generates a narrowband light observation image from the second imaging signal. The image processing unit 402 includes an image generation unit 402a, a thermally-denatured region extraction unit 402b, a blood vessel region extraction unit 402c, an adjustment unit 402d, a calculation unit 402e, and an output unit 402f.

The image generation unit 402a generates a fluorescence image from a first imaging signal obtained by capturing fluorescence produced by irradiating excitation light from the second light source unit 32. In addition, the image generation unit 402a generates a narrowband light observation image from a second imaging signal captured by irradiating narrow band light from the first light source unit 31.

The thermally-denatured region extraction unit 402b extracts the thermally-denatured region from the fluorescence image obtained by irradiating the biological tissue with the excitation light and imaging the fluorescence. The thermally-denatured region extraction unit 402b extracts, as a thermally-denatured region, a region having luminance equal to or higher than a threshold value due to fluorescence generated by AGEs in a fluorescence image captured by irradiating a biological tissue with excitation light.

The blood vessel region extraction unit 402c extracts a blood vessel region from a narrowband light observation image captured by irradiating a biological tissue with narrow band light having a wavelength determined according to an absorption rate of hemoglobin. For example, since amber light has a higher hemoglobin absorption rate than red light, a longer wavelength than green light, and light reaches a deep portion, deep blood vessels can be more easily observed than observation with normal light. The blood vessel region extraction unit 402c extracts, as a deep blood vessel region, an area having luminance of amber light equal to or less than a threshold due to absorption by hemoglobin in the narrowband light observation image captured by irradiating amber light.

The adjustment unit 402d aligns the fluorescence image and the narrowband light observation image. The adjustment unit 402d performs alignment such that positions of a feature point (characteristic points of the image, for example, the edge of the lesion or bleeding point) in the fluorescence image and a feature point in the narrowband light observation image correspond to each other. In addition, the adjustment unit 402d may extract feature information in a first reference image captured by irradiation with reference light under an imaging condition for capturing the fluorescence image and a second reference image captured by irradiation with the reference light under an imaging condition for capturing the narrowband light observation image, and perform the alignment between the fluorescence image and the narrowband light observation image on the basis of the feature information, the reference light having a wavelength different from the narrowband light used when obtaining the fluorescence image and being light narrowband light. The wavelength of the reference light is not particularly limited. Furthermore, the feature information is, for example, position information of a feature point.

The calculation unit 402e calculates the distance between the thermally-denatured region and the blood vessel region.

The output unit 402f outputs information corresponding to the distance between the thermally-denatured region and the blood vessel region. The output unit 402f outputs a display control signal in which, for example, the distance between the thermally-denatured region and the blood vessel region is superimposed on a display image to be displayed on the display device 5. In addition, the output unit 402f may output information notifying that the distance between the thermally-denatured region and the blood vessel region is equal to or less than a threshold value. For example, in a case where the distance between the thermally-denatured region and the blood vessel region is equal to or less than a threshold value, the output unit 402f may output a display control signal for superimposing and notifying a warning by a color or a mark on a display image to be displayed on the display device 5.

The input unit 403 receives inputs of various operations related to the endoscope system 1 and outputs the received operations to the control unit 405. The input unit 403 includes a mouse, a foot switch, a keyboard, a button, a switch, a touch panel, and the like.

The recording unit 404 is realized by using a recording medium such as a volatile memory, a nonvolatile memory, a solid state drive (SSD), a hard disk drive (HDD), or a memory card. The recording unit 404 records data including various parameters and the like necessary for the operation of the endoscope system 1. Furthermore, the recording unit 404 includes a program recording unit 404a that records various programs for operating the endoscope system 1.

The control unit 405 is realized by using a processor having hardware such as an FPGA or a CPU, and a memory that is a temporary storage area used by the processor. The control unit 405 integrally controls each unit constituting the endoscope system 1.

### Processing of Control Device

Next, processing executed by the control device 4 will be described.

FIG. 4 is a view illustrating an example of a biological tissue of a subject. As illustrated in FIG. 4, the subject has a blood vessel region A1. In addition, when thermal treatment such as lesion excision is performed on a surface S of the biological tissue of the subject by the energy device, the excised surface is denatured by heat, and a thermally-denatured region A2 is formed. At this time, processing in which the control device 4 causes the display device 5 to display distance L1 between the blood vessel region A1 and the thermally-denatured region A2 in the horizontal direction (direction along the surface S) will be described.

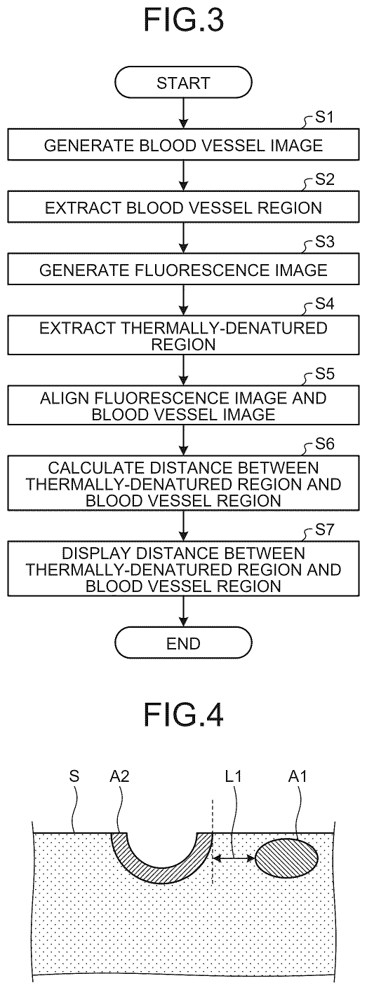

FIG. 3 is a flowchart illustrating an outline of processing executed by the control device. As illustrated in FIG. 3, first, the image generation unit 402a generates a narrowband light observation image from the second imaging signal captured by irradiating the biological tissue with narrow band light from the first light source unit 31 (step S1).

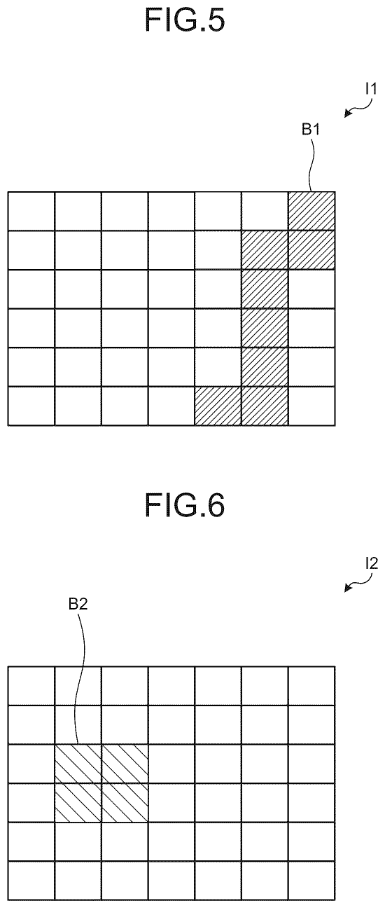

Subsequently, the blood vessel region extraction unit 402c extracts a blood vessel region from the narrowband light observation image generated by the image generation unit 402a (step S2). FIG. 5 is a diagram illustrating an example of the narrowband light observation image. As illustrated in FIG. 5, the blood vessel region extraction unit 402c extracts pixels regarded as a hatched blood vessel region B1 in a narrowband light observation image I1. In the narrowband light observation image I1, the blood vessel region extraction unit 402c extracts, as the blood vessel region B1, pixels having luminance equal to or lower than a threshold due to absorption by hemoglobin.

Thereafter, the image generation unit 402a generates a fluorescence image from the first imaging signal obtained by capturing fluorescence produced by irradiating the biological tissue with excitation light from the second light source unit 32 (step S3).

Furthermore, the thermally-denatured region extraction unit 402b extracts the thermally-denatured region from the fluorescence image obtained by capturing fluorescence produced by irradiating the biological tissue with the excitation light (step S4). FIG. 6 is a view illustrating an example of the fluorescence image. As illustrated in FIG. 6, the thermally-denatured region extraction unit 402b extracts pixels regarded as the thermally-denatured region B2 hatched in a fluorescence image I2. The thermally-denatured region extraction unit 402b extracts, as the thermally-denatured region B2, pixels having luminance equal to or higher than a threshold value due to fluorescence by AGEs in the fluorescence image I2.

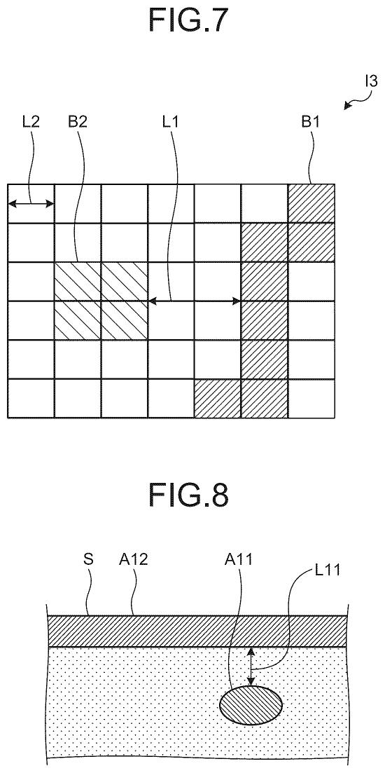

Subsequently, the adjustment unit 402d aligns the narrowband light observation image I1 and the fluorescence image I2 (step S5). FIG. 7 is a view illustrating an image created by superimposing a narrowband light observation image and a fluorescence image. As illustrated in FIG. 7, the adjustment unit 402d generates a superimposed image I3 in which the narrowband light observation image I1 and the fluorescence image I2 are superimposed so that the positions of the feature point in the narrowband light observation image I1 and the feature point in the fluorescence image I2 overlap.

Furthermore, the calculation unit 402e calculates the distance between the thermally-denatured region B2 and the blood vessel region B1 (step S6). Distance L2 corresponding to the size of one pixel of the superimposed image I3 can be calculated by detecting the distance between the distal end of the endoscope 2 and the surface S of the biological tissue of the subject with a distance sensor or the like. Then, since the distance L1 between the blood vessel region B1 and the thermally-denatured region B2 corresponds to the length of two pixels, the calculation unit 402e calculates the distance L1 using the distance L2. That is, the calculation unit 402e estimates the actual distance between the thermally-denatured region and the blood vessel of the subject from the distance L1 between the blood vessel region B1 and the thermally-denatured region B2 in the superimposed image I3.

Then, the output unit 402f outputs information corresponding to the distance between the thermally-denatured region B2 and the blood vessel region B1 (step S7). The output unit 402f outputs a display control signal that displays, for example, the distance between the thermally-denatured region B2 and the blood vessel region B1 on the display device 5.

According to the endoscope system 1 described above, since the information corresponding to the distance between the thermally-denatured region B2 and the blood vessel region B1 is output on the basis of the narrowband light observation image I1 and the fluorescence image I2, an operator can easily recognize the distance between the region where the thermal treatment is performed and the blood vessel.

### Modified Example

FIG. 8 is a view illustrating an example of the biological tissue of the subject. As illustrated in FIG. 8, a blood vessel region A11 exists in a deep portion of the subject. In addition, when thermal treatment such as lesion excision is performed on the surface S of the biological tissue of the subject by the energy device, the excised surface is denatured by heat, and a thermally-denatured region A12 is formed. At this time, the control device 4 may cause the display device 5 to display the distance L11 between the blood vessel region A11 and the thermally-denatured region A12 in the depth direction. The depth direction means a direction orthogonal to the surface S of the biological tissue.

The calculation unit 402e calculates the depth of the thermally-denatured region A12 from the fluorescence image. Since there is a correlation between the depth of the thermally-denatured region A12 and the luminance of the fluorescence image, the calculation unit 402e can estimate the depth of the thermally-denatured region A12 from the luminance of the fluorescence image on the basis of the correlation obtained in advance by measurement.

In addition, the calculation unit 402e extracts the depth of the blood vessel region A11 from the narrowband light observation image. The calculation unit 402e extracts a deep blood vessel region from a first narrowband light observation image captured by irradiation with amber light as narrowband light, extracts a middle blood vessel region from a second narrowband light observation image captured by irradiation with green light as narrowband light, and extracts a surface blood vessel region from a third narrowband light observation image captured by irradiation with blue-violet light as narrowband light. Then, the calculation unit 402e can estimate the depth of the blood vessel region A11 from the blood vessel regions of the deep layer to the surface layer.

Then, the calculation unit 402e calculates the distance between the thermally-denatured region A12 and the blood vessel region A11 in the depth direction.

Further, the output unit 402f outputs information corresponding to the distance L11 between the thermally-denatured region A12 and the blood vessel region A11. The output unit 402f outputs a display control signal that displays, for example, the distance L11 between the thermally-denatured region A12 and the blood vessel region A11 in the depth direction on the display device 5.

According to the modification described above, since the information corresponding to the distance L11 between the thermally-denatured region A12 and the blood vessel region A11 in the depth direction is output on the basis of the narrowband light observation image and the fluorescence image, the operator can easily recognize the distance between the region where the thermal treatment is performed and the blood vessel.

Additionally, the adjustment unit 402d may extract feature information in a first reference image captured by irradiation with reference light under an imaging condition for capturing the fluorescence image and a second reference image captured by irradiation with the reference light under an imaging condition for capturing the narrowband light observation image, and perform the alignment between the fluorescence image and the narrowband light observation image on the basis of the feature information, the reference light having a wavelength different from the narrowband light used when obtaining the fluorescence image and being light narrowband light. The wavelength of the reference light is not particularly limited. Furthermore, the feature information is, for example, position information of a feature point. By performing alignment between the fluorescence image and the narrowband light observation image by the image captured by irradiation with the reference light, the alignment accuracy can be improved.

In addition, the blood vessel region extraction unit 402c may extract a deep blood vessel region from the first narrowband light observation image captured by irradiation with amber light as narrowband light, extracts a middle blood vessel region from the second narrowband light observation image captured by irradiation with green light as narrowband light, and extracts a surface blood vessel region from the third narrowband light observation image captured by irradiation with blue-violet light as narrowband light.

At this time, the output unit 402f outputs information corresponding to at least two distances selected from the distance between the thermally-denatured region and the deep blood vessel region, the distance between the thermally-denatured region and the middle blood vessel region, and the distance between the thermally-denatured region and the surface blood vessel region. As a result, the operator can recognize the distance between the thermally-denatured region and the blood vessel region in two or more selected layers among the deep layer to the surface layer.

In addition, the output unit 402f may output information corresponding to one distance selected from the distance between the thermally-denatured region and the deep blood vessel region, the distance between the thermally-denatured region and the middle blood vessel region, and the distance between the thermally-denatured region and the surface blood vessel region. As a result, the operator can recognize the distance between the thermally-denatured region and the blood vessel region in one selected layer of the deep layer to the surface layer.

Furthermore, the control unit 405 may have a function as a learning unit of the learning device. The control unit 405 may generate a learned model by performing machine learning using teacher data in which a fluorescence image obtained by capturing fluorescence produced by irradiating a biological tissue with excitation light and a narrowband light observation image captured by irradiating the biological tissue with narrowband light having a wavelength determined according to an absorption rate of hemoglobin to image are used as input data and information according to distance between a thermally-denatured region extracted from the fluorescence image and a blood vessel region extracted from the narrowband light observation image is output as output data. Here, the learned model includes a neural network in which each layer has one or a plurality of nodes. In addition, the type of machine learning is not particularly limited, but for example, it is sufficient that teaching data and learning data in which fluorescence images and narrowband light observation images of a plurality of subjects are associated with distances between thermally-denatured regions and blood vessel regions calculated from the plurality of fluorescence images and the narrowband light observation images are prepared, and the learning is performed by inputting the teaching data and the learning data to a calculation model based on a multilayer neural network. Further, as a machine learning method, for example, a method based on a deep neural network (DNN) of a multilayer neural network such as a convolutional neural network (CNN) or a 3D-CNN is used. Furthermore, as a machine learning method, a method based on a recurrent neural network (RNN), long short-term memory (LSTM) units obtained by extending the RNN, or the like may be used. Note that a learning unit of a learning device different from the control device 4 may execute these functions.

According to the disclosure, it is possible to realize an assistance device, an operation method of the assistance device, an operation program of the assistance device, a medical system, and a learning device capable of easily recognizing the distance between a region subjected to thermal treatment and a blood vessel.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An assistance device comprising:
a processor comprising hardware, the processor being configured to:
acquire a fluorescence image obtained by capturing fluorescence produced by irradiating a biological tissue with excitation light, and a narrowband light observation image captured by irradiating the biological tissue with first narrowband light having a wavelength determined according to an absorption rate of hemoglobin;
extract a thermally-denatured region from the fluorescence image;
extract a blood vessel region from the narrowband light observation image;
perform alignment between the fluorescence image and the narrowband light observation image; and
output information corresponding to a distance between the thermally-denatured region and the blood vessel region.

2. The assistance device according to claim 1, wherein the excitation light has a wavelength that excites a substance contained in the thermally-denatured region.

3. The assistance device according to claim 1, wherein the first narrowband light is amber light.

4. The assistance device according to claim 1, wherein the first narrowband light is blue-violet light.

5. The assistance device according to claim 1, wherein the first narrowband light is green light.

6. The assistance device according to claim 1, wherein the processor is configured to calculate the distance between the thermally-denatured region and the blood vessel region.

7. The assistance device according to claim 1, wherein the processor is configured to
extract feature information in a first reference image captured by irradiation with reference light under an imaging condition for capturing the fluorescence image and a second reference image captured by irradiation with the reference light under an imaging condition for capturing the narrowband light observation image, the reference light being second narrowband light having a different wavelength from the first narrowband light, and
perform the alignment between the fluorescence image and the narrowband light observation image based on the feature information.

8. The assistance device according to claim 7, wherein
the processor is configured to
extract a deep blood vessel region from a first narrowband light observation image captured by irradiation with amber light as the first narrowband light,
extract a middle blood vessel region from a second narrowband light observation image captured by irradiation with green light as the first narrowband light, and
extract a surface blood vessel region from a third narrowband light observation image captured by irradiation with blue-violet light as the first narrowband light.

9. The assistance device according to claim 8, wherein the processor is configured to output information corresponding to at least two distances selected from a distance between the thermally-denatured region and the deep blood vessel region, a distance between the thermally-denatured region and the middle blood vessel region, and a distance between the thermally-denatured region and the surface blood vessel region.

10. The assistance device according to claim 8, wherein the processor is configured to output information corresponding to one distance selected from a distance between the thermally-denatured region and the deep blood vessel region, a distance between the thermally-denatured region and the middle blood vessel region, and a distance between the thermally-denatured region and the surface blood vessel region.

11. The assistance device according to claim 6, wherein
the processor is configured to
extract a pixel regarded as the thermally-denatured region in the fluorescence image,
extract a pixel regarded as the blood vessel region in the narrowband light observation image, and
calculate a shortest distance between the pixel regarded as the thermally-denatured region and the pixel regarded as the blood vessel region.

12. The assistance device according to claim 6, wherein
the processor is configured to
calculate a depth of the thermally-denatured region from the fluorescence image,
extract a depth of the blood vessel region from the narrowband light observation image, and
calculate a distance between the thermally-denatured region and the blood vessel region in a depth direction.

13. The assistance device according to claim 1, wherein the processor is configured to superimpose information corresponding to the distance between the thermally-denatured region and the blood vessel region on a display image.

14. The assistance device according to claim 1, wherein the processor is configured to output a display control signal that displays the distance between the thermally-denatured region and the blood vessel region on a display.

15. The assistance device according to claim 1, wherein the processor is configured to output information notifying that the distance between the thermally-denatured region and the blood vessel region is equal to or less than a threshold value.

16. A method of operating an assistance device, comprising:
extracting a thermally-denatured region from a fluorescence image obtained by capturing fluorescence produced by irradiating a biological tissue with excitation light;
extracting a blood vessel region from a narrowband light observation image captured by irradiating the biological tissue with narrowband light having a wavelength determined according to an absorption rate of hemoglobin;
performing alignment between the fluorescence image and the narrowband light observation image; and
outputting information corresponding to a distance between the thermally-denatured region and the blood vessel region.

17. A non-transitory computer-readable recording medium on which an executable program is recorded, the program causing an assistance device to execute:
extracting a thermally-denatured region from a fluorescence image obtained by capturing fluorescence produced by irradiating a biological tissue with excitation light;
extracting a blood vessel region from a narrowband light observation image captured by irradiating the biological tissue with narrowband light having a wavelength determined according to an absorption rate of hemoglobin;
performing alignment between the fluorescence image and the narrowband light observation image; and
outputting information corresponding to a distance between the thermally-denatured region and the blood vessel region.

18. A medical system comprising:
a light source configured to irradiate a biological tissue with excitation light and irradiate the biological tissue with narrowband light having a wavelength determined according to an absorption rate of hemoglobin;
an endoscope configured to generate a first imaging signal obtained by capturing fluorescence produced by irradiating the biological tissue with the excitation light and a second imaging signal captured by irradiating the biological tissue with the narrowband light; and
an image processing device comprising a processor comprising hardware, the processor being configured to:
generate a fluorescence image from the first imaging signal;
generate a narrowband light observation image from the second imaging signal;
extract a thermally-denatured region from the fluorescence image;
extract a blood vessel region from the narrowband light observation image;
perform alignment between the fluorescence image and the narrowband light observation image; and
output information corresponding to a distance between the thermally-denatured region and the blood vessel region.

19. A learning device comprising:
a processor comprising hardware, the processor being configured to
generate a learned model by performing machine learning using teacher data in which a fluorescence image obtained by capturing fluorescence produced by irradiating a biological tissue with excitation light and a narrowband light observation image captured by irradiating the biological tissue with narrowband light having a wavelength determined according to an absorption rate of hemoglobin are used as input data, alignment between a thermally-denatured region extracted from the fluorescence image and a blood vessel region extracted from the narrowband light observation image is performed, and information according to a distance between the thermally-denatured region and the blood vessel region after performing the alignment is output as output data.
